(19) United States
(12) Patent Application Publication
(10) Pub. No.: US 2026/0224384 A1
(43) Pub. Date: Aug. 6, 2026

(54) CUSTOMIZED HINGE FOR CONTROLLING EXTENSION AND FLEXION MOTION OF AN ORTHOSIS DURING USE

(71) Applicant: Aspen Medical Products, LLC, Irvine, CA (US)

(72) Inventors: Mark Caniga, Huntington Beach, CA (US); Jane Price, Anaheim, CA (US); Joel Perez, Long Beach, CA (US); Albert V. Romo, Lakewood, CA (US)

(21) Appl. No.: 19/456,962

(22) Filed: Jan. 22, 2026

Related U.S. Application Data
(60) Provisional application No. 63/748,846, filed on Jan. 23, 2025.

(51) Int. Cl. A61F 5/01

(52) U.S. Cl. CPC A61F 5/0125; A61F 2005/0139; A61F 2005/0144; A61F 2005/0158; A61F 2005/0165

(57) ABSTRACT

A hinge featuring a first hinge cover and a second hinge cover that can be utilized by an orthosis. Moveable in a first and second rotational direction, the first hinge cover includes a first base component and a first arm component extending therefrom. The second hinge cover includes a second base component and a second arm component extending from the second base component. Like the first hinge cover, the second hinge cover is configured to rotate in the first rotational direction and the second rotational direction. Concurrent rotational movement of the first hinge cover and the second hinge cover in the first rotational direction places a central unit, inclusive of the hinge covers and any removable stop component, into a locked state and concurrent rotational movement of the first hinge cover and the second hinge cover in the second rotational direction places the central unit into an unlocked state.

### CROSS-REFERENCE TO RELATED APPLICATIONS

This Application claims the benefit of priority on U.S. Patent Application No. 63/748,846 filed Jan. 23, 2025, the entire contents of which are incorporated by reference herein.

### FIELD

Embodiments of the disclosure relate to the field of orthopedic braces. More specifically, one embodiment of the disclosure relates to a customized, adjustable hinge configured to control extension and flexion movement of an orthopedic brace (orthosis) during use.

### GENERAL BACKGROUND

The following description includes information that may be useful in understanding the described invention. It is not an admission that any of the information provided herein is prior art or relevant to the presently claimed invention, or that any publication specifically or implicitly referenced is prior art.

A hinged knee brace is a type of orthopedic device designed to provide support and stability to the knee joint. It features a hinge assembly, inclusive of a polycentric hinge, positioned on the medial or lateral or both sides of the knee. The polycentric hinge tracks knee motion and has features to limit range of motion. For example, the polycentric hinge may feature fixed and integrated stops designed to control the degree of flexion and extension to limit range of motion to prevent injury to a healthy knee or to prevent reinjury.

Conventional polycentric hinges, commonly used in knee braces, feature a number of disadvantages. First, conventional polycentric hinges tend to have complex architectures with multiple moving parts and a geared extension/flexion adjustment system, which are costly to manufacture and more prone to failure caused by regular usage. In many cases, conventional polycentric hinges may require regular maintenance, such as lubrication and cleaning, to ensure smooth operation. Secondly, the conventional polycentric hinges are heavy and bulky, where the added weight can be uncomfortable for some users when the brace is worn for extended periods of time and the bulkiness of the hinge is difficult to wear under clothing garments. A simpler and easier to use more cost effective hinge design for all types of orthoses, including knee braces, is needed.

### BRIEF DESCRIPTION OF THE DRAWINGS

Embodiments of the invention are illustrated by way of example and not by way of limitation in the figures of the accompanying drawings, in which like references indicate similar elements and in which:

FIG. 1 is a perspective view of an exemplary embodiment of an orthosis represented as hinged knee brace including a customized hinge located on at least a medial side or a lateral or both sides of the hinged knee brace.

FIG. 2 is a first illustrative embodiment of mechanical components of a hinge for the orthosis of FIG. 1, including a hinge and a pair of members (e.g., struts) coupled to the hinge.

FIG. 3A is an overhead planar view of a first exemplary embodiment of the hinge of FIGS. 1-2 placed into a partially open (or unlocked) state.

FIG. 3B is an overhead planar view of the hinge of FIG. 3A placed into a closed (locked) state.

FIG. 3C is a laterally oriented perspective view of the hinge of FIG. 3A.

FIG. 4A is a perspective view of an illustrative embodiment of the hinge of FIG. 2 oriented in a resting (non-flexed) state and the hinge cover placed in an open (or unlocked) state.

FIG. 4B is a perspective view of an illustrative embodiment of the hinge of FIG. 4A with the hinge cover placed in a closed (locked) state.

FIGS. 4C-4E are perspective views illustrating an embodiment of the hinge of FIG. 4A oriented in a resting (non-flexed) state with the hinge cover transitioning from a closed (locked) state to an open (or unlocked) state followed by removal of an extension stop component and/or a flexion stop component from the hinge.

FIG. 5A is a cross-sectional view of the hinge of FIG. 4B with a first type of extension stop component operating with the struts to allow for a prescribed degree of extension.

FIG. 5B is a first illustrative embodiment of different types of extension stop components that provide different degrees of controlled extension ranging from highest-to-lowest degrees of controlled extension.

FIG. 5C is a cross-sectional view of the hinge of FIG. 4B with a first type of flexion stop component operating with the struts to allow for a prescribed degree of flexion.

FIG. 5D is a first illustrative embodiment of different types of flexion stop components that provide different degrees of controlled flexion ranging from lowest-to-highest degrees of controlled flexion.

FIG. 6A is a side perspective view of an exemplary embodiment of a first hinge cover of the hinge, including a flange formed within a portion of the first hinge cover to assist in securing the flexion/extension stop components within a prescribed area of the hinge.

FIG. 6B is a cross-sectional view of a portion of the first hinge cover including first hinge cover along with the flange member.

FIG. 7A is an overhead planar view of a second exemplary embodiment of the hinge of FIGS. 1-2 placed into a partially open (or unlocked) state.

FIG. 7B is an overhead planar view of the hinge of FIG. 7A placed into a closed (locked) state.

FIG. 7C is a laterally oriented perspective view of the hinge of FIG. 7A.

FIG. 7D is a cross-sectional view of the hinge of FIG. 7B illustrating the supplementary interior sidewalls extending from inner walls of the hinge covers for additional assistance in retention when placed into the closed (locked) state.

FIG. 8A-8E are translucent views of the hinge with extension stop components set at zero degree, fifteen degree, thirty degree, forty-five degree, and sixty degree extension ranges.

FIG. 9A-9B are translucent views of the hinge with flexion stop components set at ten degree, thirty degree, forty-five degree, and sixty degree flexion ranges.

FIG. 10 is an illustrative embodiment of a method of operation for setting the hinge into an operational state with a prescribed degree of extension and/or flexion.

FIG. 11 is a second illustrative embodiment of mechanical components of a hinge for the orthosis of FIG. 1, including a hinge and a pair of geared members (e.g., struts) coupled to the hinge.

FIG. 12A is a perspective view of an illustrative embodiment of the hinge of FIG. 11 oriented in a resting (non-flexed) state and the hinge covers of the central unit are placed in an open (or unlocked) state.

FIG. 12B is an upward perspective view of an illustrative embodiment of a backside of the first hinge cover of the central unit of FIG. 12A transition from an unlocked state to a locked state in which a cover recess is positioned over the first stability element of the extension stop component.

FIG. 12C is a planar view of an illustrative embodiment of the backside of the first hinge cover of the central unit of FIG. 12B.

FIG. 13A is a cross-sectional view of the hinge of FIG. 12A with a first type of extension stop component.

FIG. 13B is a cross-sectional view of the hinge of FIG. 13A with a selected type of extension stop component operating with the geared struts to allow for a prescribed degree of extension.

FIG. 13C is a second illustrative embodiment of different types of extension stop components that provide different degrees of controlled extension ranging from highest-to-lowest degrees of controlled extension.

FIG. 13D is a cross-sectional view of the hinge of FIG. 12A with a selected type of flexion stop component operating with the struts to allow for a prescribed degree of flexion.

FIG. 13E is a second illustrative embodiment of different types of flexion stop components that provide different degrees of controlled flexion ranging from lowest-to-highest degrees of controlled flexion.

FIG. 14 is a translucent view of the hinge of FIG. 11 with an extension stop component set at a fifteen degree extension range.

FIG. 15 is a translucent views of the hinge of FIG. 11 with a flexion stop component set at a sixty degree flexion range.

### DETAILED DESCRIPTION

Embodiments of the present disclosure generally relate to an orthosis, such as a hinged knee brace for example, which features a hinge positioned on a lateral or medial or both sides of the orthosis. The hinge includes (i) a pair of members for attachment to a cuff or other mechanical component near the entry and exit points of the orthosis and (ii) a central unit to control movement of the pair of members by at least controlling the degree of extension and/or flexion permitted by the orthosis. The pair of members may include a plurality of strut members, where the strut members may feature a proximal end having a gear (e.g., a wheel-like mechanical element with uniformly spaced teeth around its outer edge) or a smooth-edge proximal end.

According to one embodiment of the disclosure, the central unit is adapted to receive a removable extension stop component to control the degree of extension permitted by the orthosis and/or a removable flexion stop component to control the degree of flexion permitted by the orthosis. Herein, for this embodiment of the disclosure, the central unit includes a pair of hinge covers with collinear axes of rotation. The hinge covers are arranged in a first rotational position when the central unit (e.g., the hinge covers and stop component(s)) is placed into a closed (locked) state to retain the extension stop component and/or the flexion stop component and in a second rotational position when the central unit is placed into an open (or unlocked) state to allow for insertion or removal of the extension stop component and/or the flexion stop component. Rotationally coupled to a hinge front plate by a first fastening element establishing a first axis of rotation, a first hinge cover features a first attachment region surrounding an aperture to receive the first fastening element and a second attachment region constituting a distal portion of the first hinge cover. Similarly, rotationally coupled to the hinge front plate by a second fastening element establishing a second axis of rotation, a second hinge cover features a third attachment region surrounding an aperture to receive the second fastening element and a fourth attachment region constituting a distal portion of the second hinge cover.

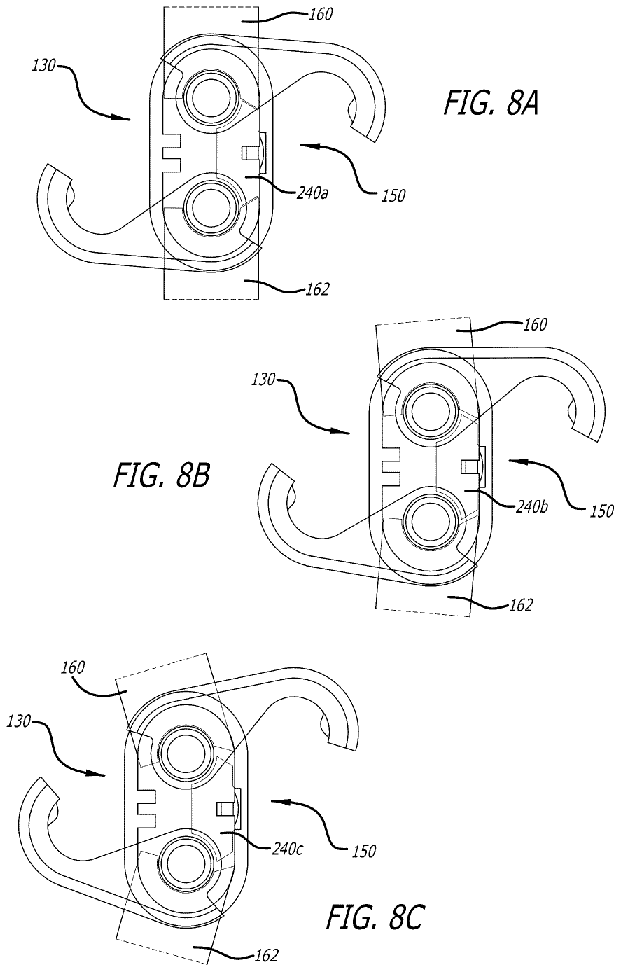

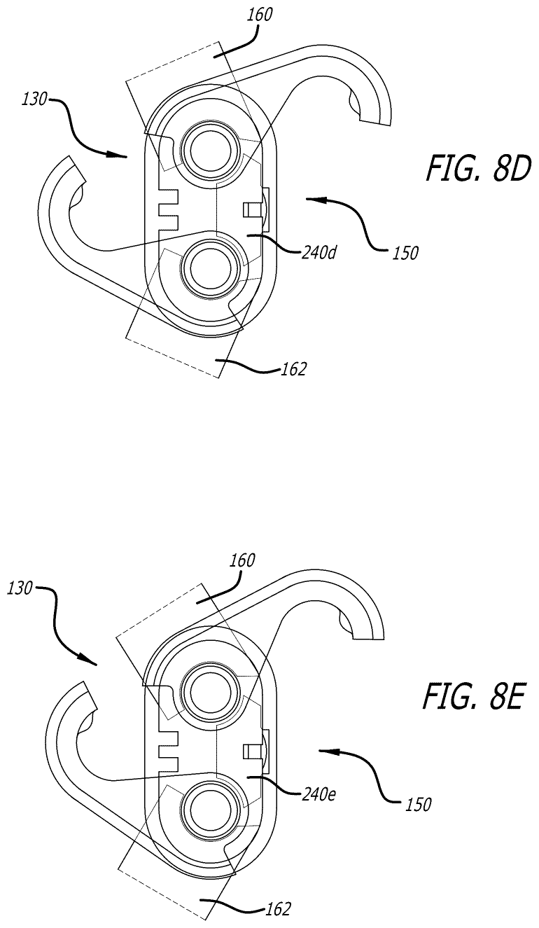

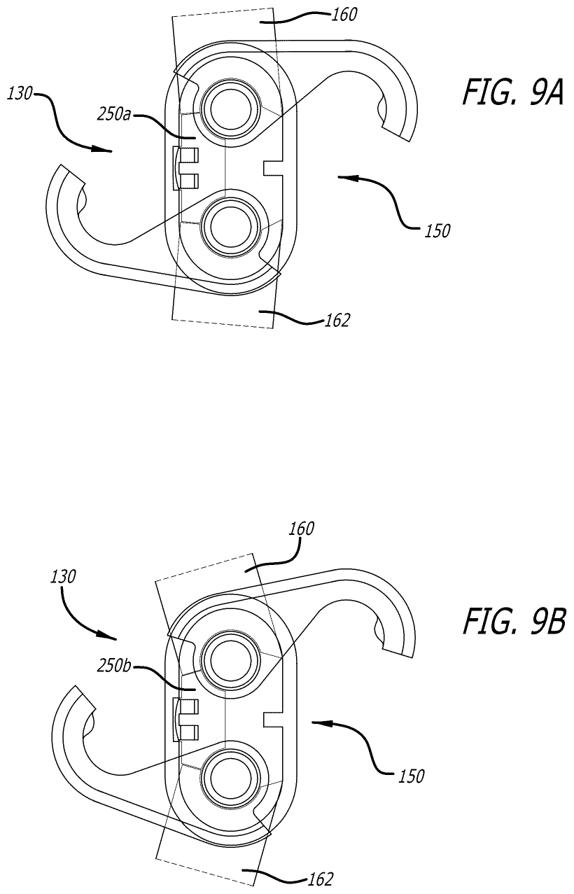

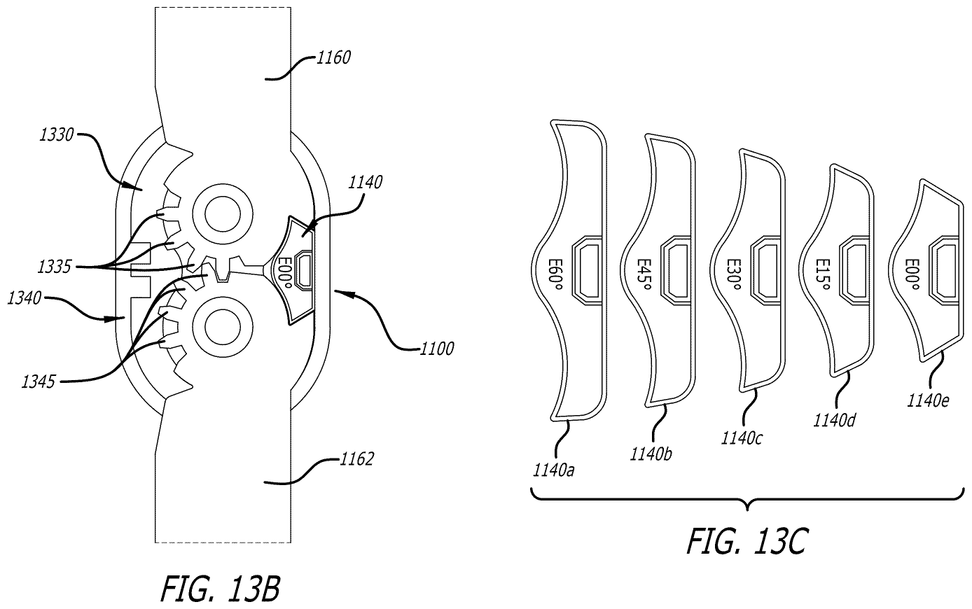

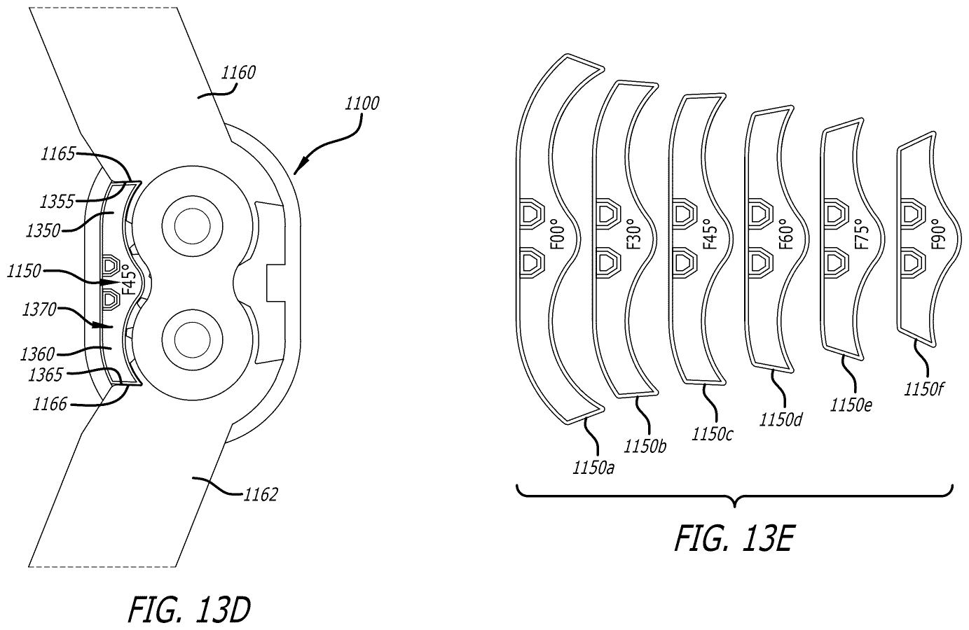

For this embodiment of the disclosure, the extension stop component is formed to reside within a first (anterior) spacing formed between the members and the hinge front/back plates. The extension stop component is adapted with a first stability element positioned on a side surface of the extension stop component for engagement with a stability notch formed along a first edge of the hinge front plate. Similar in construction, the flexion stop component is formed to reside within a second (posterior) spacing formed between the members and the hinge front/back plates. As described below, the flexion stop component is adapted with one or more (e.g., two) stability elements positioned on a side surface of the flexion stop component for engagement with corresponding stability notch(es) formed along a second edge of the hinge front plate. The second edge is opposite to the first edge on the hinge front plate. The increased sizing of the extension limiter elements (e.g., “wing” protrusions) on the extension stop component corresponds to increased degrees of extension while increased sizing of the flexion limiter elements formed in the flexion stop component corresponds to decreased degrees of flexion as illustrated in FIGS. 8A-8E, FIGS. 9A-9B, and/or FIGS. 13A-13D. Stated differently, increasing the size of the extension limiter elements reduces the range of motion for extension, and similarly, increasing the size (length) of the flexion limiter element reduces the range of motion for flexion.

### I. Terminology

As to certain terminology used, the terms “attach” and “connect” as well as other verb tenses of these terms (e.g., attached, attaching, connected, connecting, etc.) may be construed as physically coupling two items together. This physical coupling may be a direct coupling of the two items using fastener(s) or an indirect coupling that relies on one or more intermediary components to which the two items are connected. Similarly, as used herein and unless the context dictates otherwise, the term “coupled” is intended to include both direct coupling and indirect coupling. Therefore, the terms “coupled to” and “coupled with” are used synonymously.

The term “fastening element” may be construed as any physical component used to attach different items together. An illustrative example of different types of fastening elements and fastening techniques may include, but are not limited or restricted to a snap, button, clasp, clip, hook, or loop element (e.g., Velcro®), buckle, dowel/cap, rivet, screw, bolt/nut, adhesive, sealant, or other components that are connected to its complementary fastener. A “member” may be construed as a physical component that provides stability such as a strut or another type of rigid component.

The term “concave” may be construed as a curvature in a first direction (e.g., upward like an interior of a circle) while the term “convex” may be construed as a curvature in a second direction (e.g., downward like an exterior of a circle”). It is contemplated that these curvatures may be alterable with a change in viewpoint, where a surface may be convex-shaped from a posterior viewpoint and concave-shaped from an anterior viewpoint. As a result, for the subject application, the viewpoints for the figures are identified.

As used herein the term “patient” includes both humans and animals, independently of whether the patient is under the care of a medical or veterinary professional. The terms “concurrent” and “contemporaneous,” as well as their other inflections or word forms. mean at least partially overlapping in time.

In some embodiments, the orthosis may be configured to have a general tubular construction (e.g., a sleeve with an open top/bottom), where the sleeve may be configured with greater rigidity at the openings or feature cuffs (e.g., hardened material positioned within the sleeve to provide physical support for tightening straps, etc.) positioned within the sleeve to enhance rigidity at these regions of the orthosis. Alternatively, the orthosis may be configured as a wrap that is placed into a general tubular construction when a first edge is wrapped around and attached to a surface of the orthosis.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Finally, the terms “or” and “and/or” or symbol “/” as used herein are to be interpreted as inclusive or meaning any one or any combination. As an example, “A, B or C” or “A, B and/or C” mean “any of the following: A; B; C; A and B; A and C; B and C; or A, B and C.” Similarly, “A/B” may be interpreted as A; B; or A and B. An exception to this definition will occur only when a combination of elements, functions, steps, or acts are in some way inherently mutually exclusive.

All methods or orthosis aspects described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Various objects, features, aspects, and advantages of the inventive subject matter will become more apparent from the following detailed description of preferred embodiments, along with the accompanying drawing figures in which like numerals represent like components.

### II. Orthosis—General Architecture

In some embodiments, the numbers expressing quantities of properties such as sizing, degrees of motion, and so forth, may be described in connection with certain embodied inventive aspect, but these quantities may be modified while still retaining the spirit and scope of the disclosed invention when the specific properties are not explicitly claimed. Accordingly, in some embodiments, the numerical parameters set forth in the written description are illustrative approximations that can vary depending upon the desired properties sought to be obtained by a particular embodiment.

Groupings of inventive embodiments disclosed herein are not to be construed as limitations. Each inventive embodiment, as well as each illustrative component or feature within an inventive embodiment, can be referred to and may be claimed individually or in any combination with other members of the group or other elements found herein. One or more components or features can be assembled as a group of components or features associated with an embodiment of an invention or can be added or deleted from groups so that multiple embodiments with different sets of described components or features than those presented in an illustrative embodiment are contemplated. When any such inclusion or deletion occurs, the specification is herein deemed to contain the group as modified.

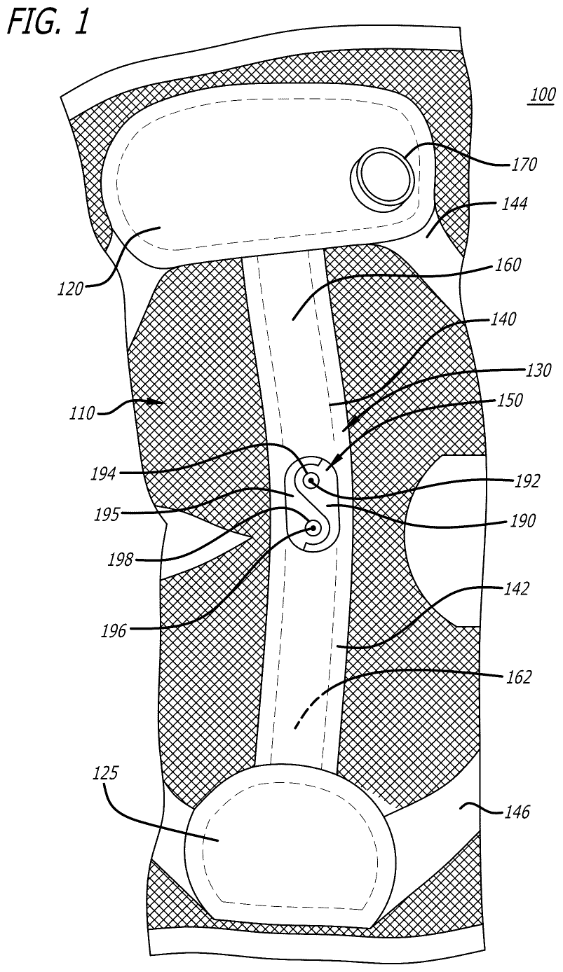

Referring to FIG. 1, a perspective view of an exemplary embodiment of an orthosis 100 operating as a hinged knee brace is shown. The orthosis 100 features a sleeve 110 adapted to support a hinge 130, which is an assembly including a central unit 150 and members (e.g., strut members) 160/162 that control movement of the orthosis 100 when worn. For this embodiment, the sleeve 110 includes pocket regions for a first cuff 120 (e.g., a sheet of hardened material positioned above the knee such as resting over the femur) and a second cuff 125 (e.g., a sheet of hardened material positioned below the knee such as resting over the tibia).

The sleeve 110 further includes channels 140 and 142 for the strut members 160 and 162 coupled to the central unit 150 and the cuffs 120/125. Additionally, the sleeve 110 further includes tunnels 144 and 146 to substantially integrate interconnects (not shown) made of inflexible and/or flexible material (e.g., wires, straps made of an elastic or inelastic material, or any combination thereof) connected between the cuffs 120/125, where the interconnects may be tightened or loosened by a rotating cable tensioning mechanism 170 accessible through the sleeve 110.

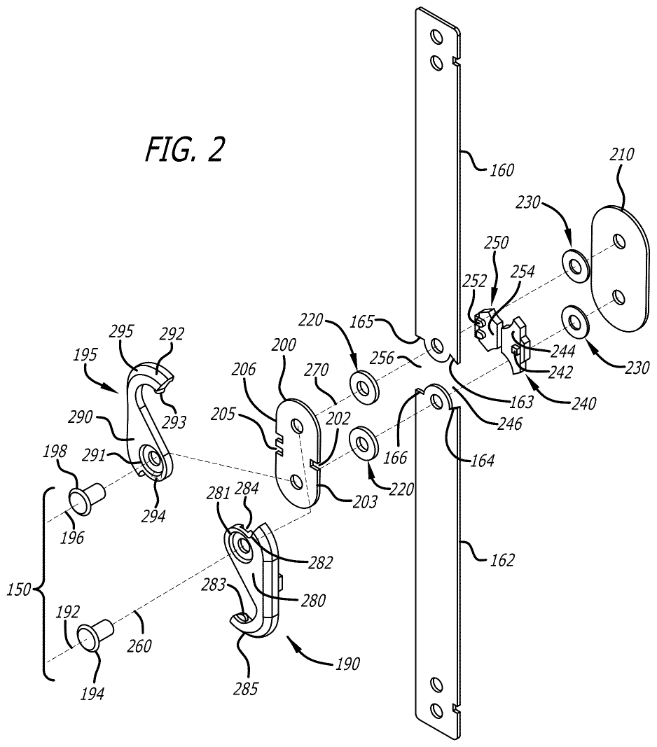

As further shown in FIG. 1, positioned on a lateral and/or medial side of the orthosis 100, the hinge 130 includes (i) the pair of strut members 160/162 for attachment to cuffs 120/125 or other mechanical component near the entry and exit points of the orthosis and (ii) the central unit 150 to control movement of the pair of strut members 160/162 by at least controlling the degree of extension and/or flexion permitted for the strut members 160/162, and thus, a body part (e.g., leg, arm, etc.) of the patient placed within orthosis 100. Although not shown, the central unit 150 is adapted to receive a removable (extension) stop component to control the degree of extension permitted by the orthosis 100 and/or a removable (flexion) stop component to control the degree of flexion permitted by the orthosis 100 (see FIG. 2). It is contemplated that the hinge 130 is operational even if a flexion stop component and/or an extension stop component are not positioned as part of the hinge 130.

For this embodiment, the central unit 150 includes a first hinge cover 190 rotational about a first axis of rotation collinear with a first fastening element 194 and a second hinge cover 195 rotational about a second axis of rotation 196 collinear with a second fastening element 198. As shown, the central unit is placed into a closed (locked) state and the axes of rotation 192/196 are collinear with each other. For placement in an open (or unlocked) state, the first hinge cover 190 would need to be rotated in a first direction (e.g., counterclockwise “CCW” direction) contemporaneously with rotation of the second hinge cover 195 in the first direction. Contemporaneous (or concurrent) movement of the hinge covers 190/195 is needed to transition from a closed (locked) state to an open (or unlocked) state, as rotation of only one of the hinge covers 190 or 195 would be ineffectual to change the operating state of the central unit 150. The dual rotation criterion ensures that extension and/or flexion stop components positioned as part of the central unit 150 cannot be dislodged or unintentionally removed as both hinge covers 190 and 195 need to be rotated to provide sufficient clearance to remove the stop components therefrom.

### III. FIRST HINGE-GENERAL ARCHITECTURE

Referring now to FIG. 2, an illustrative embodiment of mechanical components of the hinge 130 of FIG. 1 is shown, which include the central unit 150 and the pair of strut members 160 and 162. For this embodiment, configured for toolless installation and/or removal of extension/flexion stop components (described below), the central unit 150 includes the first and second fastening elements 194 and 198; the first and second hinge covers 190 and 195; a hinge front plate 200; a hinge back plate 210; a first set of washers 220 positioned between the hinge front plate 200 and the strut members 160 and 162; a second set of washers 230 positioned between the hinge back plate 210 and the strut members 160 and 162; an optional extension stop component 240; and/or an optional flexion stop component 250. A coupling path 260 illustrates an attachment sequence for the first fastening element 194 to form the first axis of rotation 192 while a second coupling path 270 illustrates an attachment sequence for the second fastening element 198 to form the second axis of rotation 196.

More specifically, the first hinge cover 190 includes a first base component 280 and a first arm component 285 while the second hinge cover 195 includes a second base component 290 and a second arm component 295. Herein, according to this embodiment of the disclosure. the first hinge cover 190 is rotationally coupled to the hinge front plate 200 by insertion of the first fastening element 194 into a recessed aperture 281 formed within the first base component 280. An end section 282 of the first base component 280 features a recessed area 284 sized to receive a detent (protrusion) 293 situated within the second arm component 295 of the second hinge cover 195. The first arm component 285 of the first hinge cover 190 includes a first detent (protrusion) 283, which is sized for retention within a recessed area 294 of the second hinge cover 195.

Similarly, the second hinge cover 195 is rotationally coupled to the hinge front plate 200 by insertion of the second fastening element 198 into a recessed aperture 291 formed within the second base component 290. An end section 292 of the second base component 290 features the recessed area 294 sized to receive the first detent 283 situated within the first arm component 285 of the first hinge cover 190. The second arm component 295 of the second hinge cover 195 includes the detent (protrusion) 293, which is sized for retention within the recessed area 284 of the first hinge cover 190.

Referring still to FIG. 2, the hinge front plate 200 is configured with a first set of stability notches (e.g., first stability notch 202) may be formed along a first edge 203 of the hinge front plate 200. The first stability notch 202 is formed to engage with a first stability element 242 positioned on a side surface 244 of the extension stop component 240. This engagement is designed to prevent (or at least substantially mitigate) vertical movement of the extension stop component 240 during usage of the orthosis 100. Additionally, a second set of stability notches (e.g., second and third stability notches 205) may be formed along a second edge 206 of the hinge front plate 200. These stability notches 205 are formed to engage with a corresponding set of stability elements 252 positioned on a side surface 254 of the flexion stop component 250. As shown, the number of stability elements 242 positioned on the extension stop component 240 may differ from the number of stability elements 252 positioned on the flexion stop component 250 for easy identification as to whether the stop component is designed to control extension or flexion.

The extension stop component 240 is positioned to reside within a first (anterior) spacing 246 formed between first edges 163 and 164 of the strut members 160 and 162, with the first stability element 242 being positioned to engage with the first stability notch 202 when the extension stop component 240 is placed within the first spacing 246. Similar in construction, the flexion stop component 250 is formed to reside within a second (posterior) spacing 256 formed between second edges 165 and 166 of the strut members 160 and 162, with the set of stability elements 252 being positioned to engage with the stability notches 205 when the flexion stop component 250 is placed within the second spacing 256.

During extension, when viewed from a lateral-to-medial perspective for a right leg, forward movement of the second strut member 162 causes rotation of the central unit 150, where the first edge 164 of the second strut member 162 engages the extension stop component 240 sized to allow a prescribed degree of extension. The first strut member 160 remains fixed relative to the central unit 150, while the second strut member 162 rotates relative to the upper strut-moving forward during extension to engage the extension stop component 240. During flexion, rearward movement of the second strut member 162 produces rotation of the central unit 150, causing the second edge 166 of the second strut member 162 to engage the flexion stop component 250, which is sized to allow a prescribed degree of flexion. The second strut member 162 rotates rearward relative to the first strut member 160 to engage the flexion stop component 250.

### IV. FIRST HINGE ARCHITECTURE AND OPERABILITY

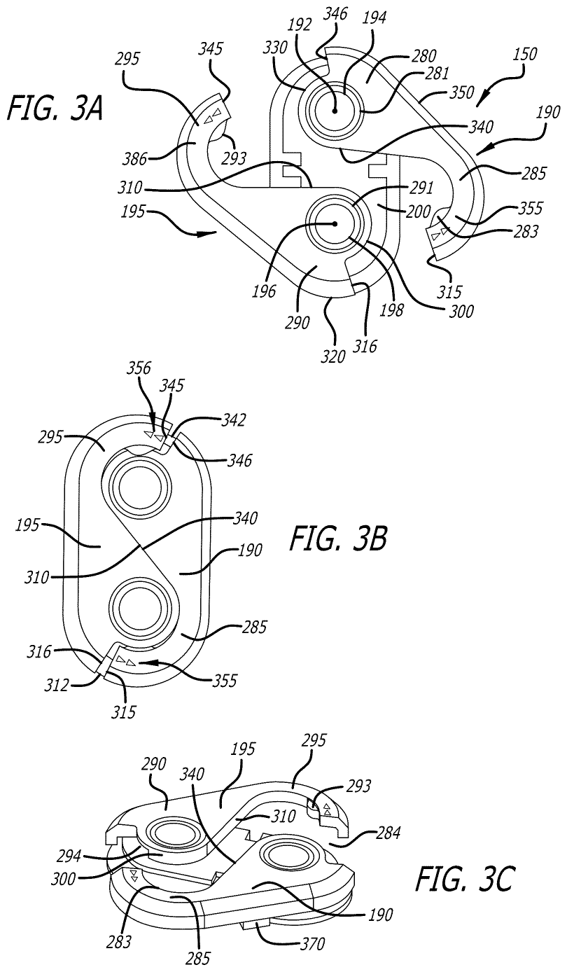

Referring now FIG. 3A, an overhead planar view of a first exemplary embodiment of the central unit 150 of FIGS. 1-2placed into a partially open (or unlocked) state is shown. Herein, the central unit 150 features the first hinge cover 190 and the second hinge cover 195. The first hinge cover 190 is mounted on the hinge front plate 200 (see FIG. 2) and rotational about the first axis of rotation 192. The first axis of rotation 192 is colinear with the first fastening element 194. Similarly, the second hinge cover 195 is mounted on the hinge front plate 200 and rotational about the second axis of rotation 196. The second axis of rotation 196 is colinear with the second fastening element 198.

As shown, the first hinge cover 190 includes the first base component 280 and the first arm component 285 while the second hinge cover 195 includes the second base component 290 and the second arm component 295. Herein, the first base component 280 corresponds to a section of the first hinge cover 190 that surrounds the aperture 281 adapted to receive the first fastening element 194. The first arm component 285, corresponding to a distal portion of the first hinge cover 190, is configured with a concave-shaped curvature to (i) partially align with an angular (convex) segment 300 of an inner edge 310 of the second hinge cover 195 and (ii) provide a gap 312 between a distal end 315 for the first arm component 285 of the first hinge cover 190 and a proximal end 316 for the second base component 290 of the second hinge cover 195 that connects the inner edge 310 with the outer edge 320.

Similarly in construction, the second base component 290 corresponds to a section of the second hinge cover 195 that surrounds the aperture 291 adapted to receive the second fastening element 198. The second arm component 295 corresponds to a section of the second hinge cover 195 having a concave-shaped curvature that (i) partially aligns with an angular segment 330 of an inner edge 340 of the first hinge cover 190 and (ii) provides a gap 342 between a distal end 345 of the second hinge cover 195 and a proximal end 346 of the first hinge cover 190 that connects the inner edge 340 with an outer edge 350.

As a result, as shown in FIG. 3B, when the central unit 150 is placed into a closed (locked) state, the inner edge 340 of the first hinge cover 190 is complementary to and aligned with the inner edge 310 of the second hinge cover 195. Also, the first gap 312 is formed between the proximal end 316 of the second hinge cover 195 and the distal end 315 of the first hinge cover 190 featuring indicia 355 identifying a direction of movement needed from the first gap 312 to unlock the central unit 150. The second gap 342 is formed between the proximal end 346 of the first hinge cover 190 and the distal end 345 of the second hinge cover 195 featuring indicia 356 identifying a direction of movement needed from the second gap 342 to unlock the central unit 150.

As shown in FIGS. 3A-3B, the inner edge 340 of the first hinge cover 190 includes the detent (protrusion) 283 positioned toward at the distal end 315 of the first arm component 285 while the inner edge 310 of the second hinge cover 195 includes a detent (protrusion) 293 positioned toward at the distal end 345 of the second arm component 295. Given that the first hinge cover 190 and the second hinge cover 195 may have mirror constructions, where one hinge cover (e.g., first hinge cover 190) is inclined inwardly and the other hinge cover (e.g., second hinge cover 195) is inclined outwardly. However, the below discussion of the formulation of the detent 283 for the first hinge cover 190 is applicable to the formulation of the detent 293 for the second hinge cover 195.

More specifically, the detent 283 is configured to facilitate attachment of the first hinge cover 190 to the second hinge cover 195 while the detent 293 is configured to facilitate attachment of the second hinge cover 195 to the first hinge cover 190. More specifically, the detent 283 is sized to reside within the recessed area 294 formed within the angular segment 300 of the inner edge 310 of the second hinge cover 195 when the central unit 150 is placed in a closed (locked) state as shown in FIGS. 3B-3C. As the central unit 150 is placed into a closed (locked) state, prior to resting with the recessed area 294, the detent 283 comes into contact with an angular segment 300 of the inner edge 310 forming part of the second base component 290, which causes a slight increase in the curvature of the first arm component 285 (e.g., flex or elongation of the concave-shaped first arm component 285) until the detent 283 clears an apex formed by the angular segment 300 and returns back to its “at rest” curvature (non-flex state) to reside within the recessed area 294 to “snap” into a closed (locked) state. The same operations occur for the detent 293 placed toward the distal end 345 of the second hinge cover 195.

Referring now to FIG. 3C, the outer edge 350 of the first hinge cover 190 includes a flange 370 positioned to overlap a portion of an entry area for the stop component 240 of FIG. 2 when the first hinge cover 190 is placed into a closed (locked) state. This overlapping flange 370 is adapted to preclude removal of the extension stop component 240, unless the central unit 150 is placed into an open (or unlocked) state in which both the first and second hinge covers 190 and 195 are rotated in a first direction (e.g., counterclockwise “CCW” direction) concurrently. Similarly, the outer edge 320 of the second hinge cover 195 features a flange (not shown) positioned to overlap a portion of an entry area for the flexion stop component 250 of FIG. 2. This overlapping flange is adapted to preclude removal of the flexion stop component 250 unless the central unit 150 is placed into an open (or unlocked) state in which both the first and second hinge covers 190 and 195 are rotated in the first direction (e.g., CCW) concurrently.

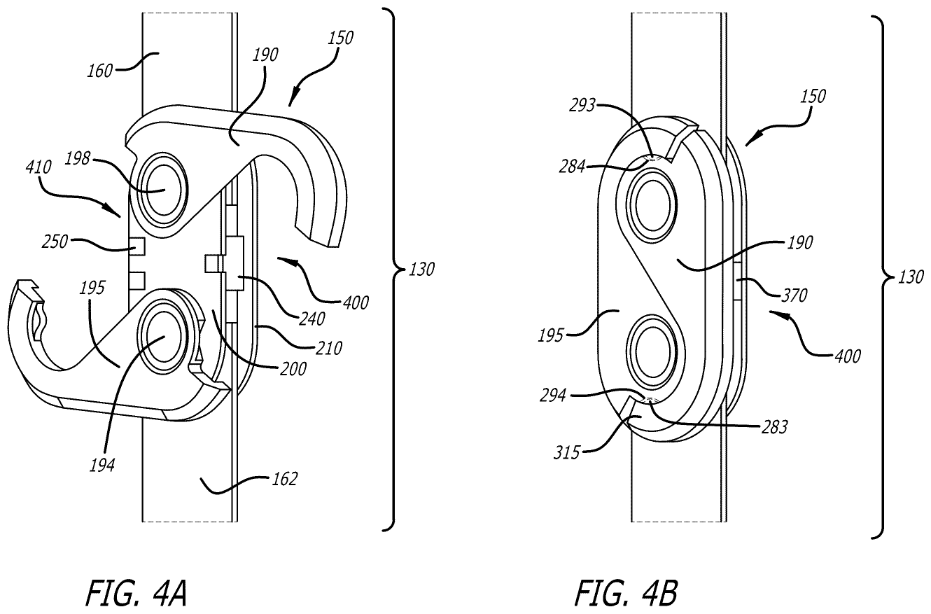

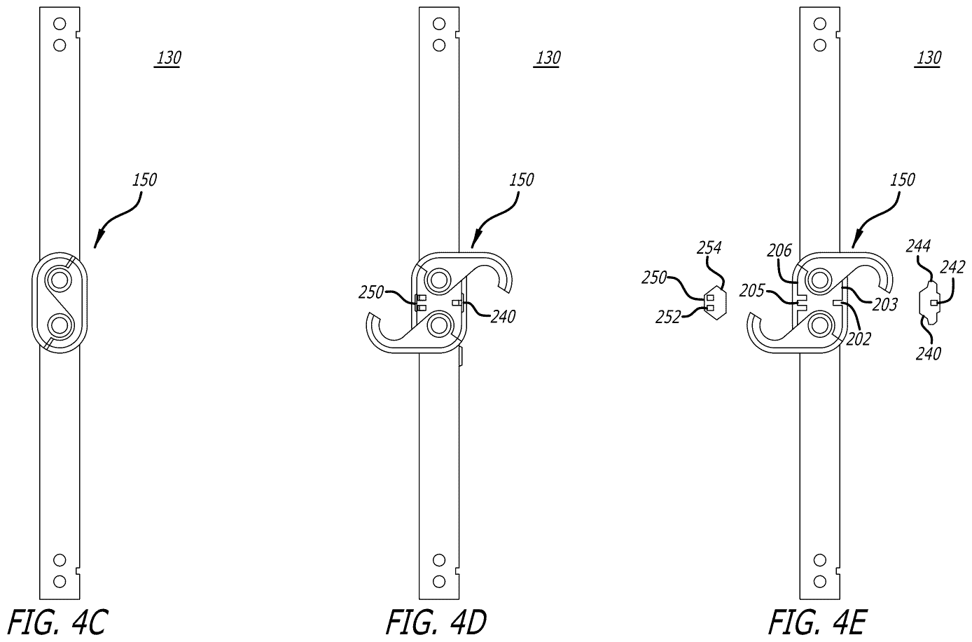

Referring now to FIG. 4A, a perspective view of an illustrative embodiment of the hinge 130 of FIG. 2 oriented in a resting (non-flexed) state and the central unit 150 placed in an open (or unlocked) state that allow the extension stop component 240 and/or the flexion stop component 250 to be installed or removed from the central unit 150, where the removal may involve replacement with a different degree of extension and/or flexion. To retain the central unit 150 to the strut members 160/162, the fastening elements 194 and 198 are placed through apertures formed in the hinge covers 190 and 195, the hinge front plate 200, strut members 160 and 162, and the hinge back plate 210. Entry areas 400 and 410 are formed between ends of the strut members 160 and 162 to receive the extension stop component 240 and/or the flexion stop component 250. The hinge covers 190 and 195 are rotated to expose the entry areas 400 and 410 for insertion of the stop components 240 and 250 when desired.

Referring to FIG. 4B, a perspective view of an illustrative embodiment of the hinge 130 of FIG. 2 oriented in a resting (non-flexed) state and the central unit 150 placed in a closed (locked) state that allow the extension stop component 240 and/or the flexion stop component 250 to be secured as part of the central unit 150. Herein, the hinge covers 190 and 195 are rotated until the detent 283 positioned towards the distal end 315 of the first hinge cover 190 is positioned to rest within the recessed area 294 formed within the second base component 290 and the detent 293 positioned towards the distal end 345 of the second hinge cover 195 is positioned to rest within the recessed area 284 similar to recessed area 294 formed within the first base component 280. As shown, the flange 370 of the first hinge cover 190 is positioned to overlap a portion of the entry area 400 to assist in securing the extension stop component 240. The same operation is performed by the flange similar positioned on the second hinge cover 195 to secure the flexion stop component 250.

Referring to FIGS. 4C-4E, perspective series of views of illustrative embodiments of the hinge 130 of prior FIGS. 4A-4B are shown, where the hinge 130 is oriented in a resting (non-flexed) state with both the extension stop component 240 and the flexion stop component 250 being removed from the central unit 150 placed in an open (or unlocked) state. The central unit 150 transitions from a closed (or locked) state (see FIG. 4C) to an open (or unlocked) state (see FIG. 4D). As shown in FIG. 4E, the hinge front plate 200 is configured with the first set of stability notches (e.g., first stability notch 202) formed along the first edge 203 and the second set of stability notches (e.g., second and third stability notches 205) may be formed along the second edge 206 of the hinge front plate 200. Upon removal of the extension stop component 240, the first stability element 242 positioned on the side surface 244 of the extension stop component 240 is disengaged from the first stability notch 202. Upon removal of the flexion stop component 250, the stability elements 252 positioned on the side surface 254 of the flexion stop component 250 are disengaged from the second and third stability notches 205. As shown, the number of stability elements 242 positioned on the extension stop component 240 may differ from the number of stability elements 252 positioned on the flexion stop component 250 for easy identification as to the type of stop component (e.g., extension or flexion), although different differentiation techniques (e.g., color, markings, etc.) may be used as well.

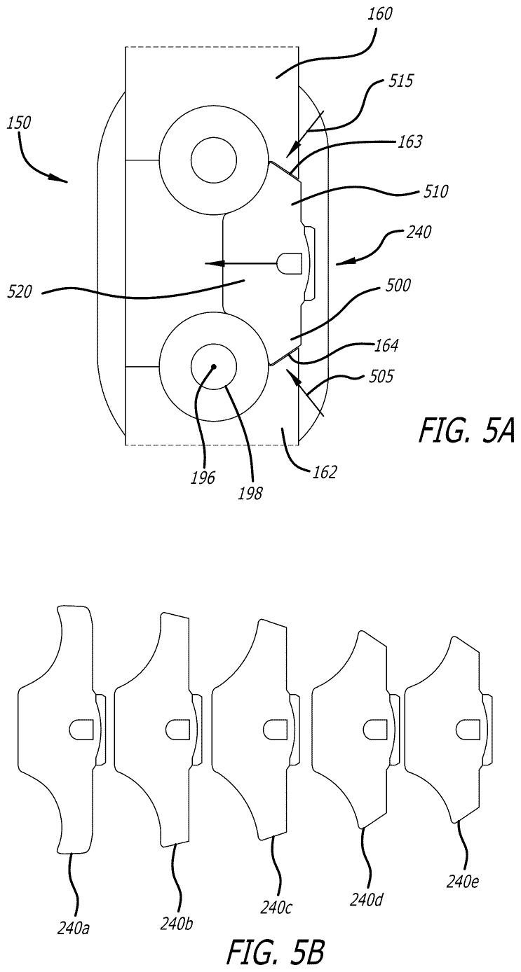

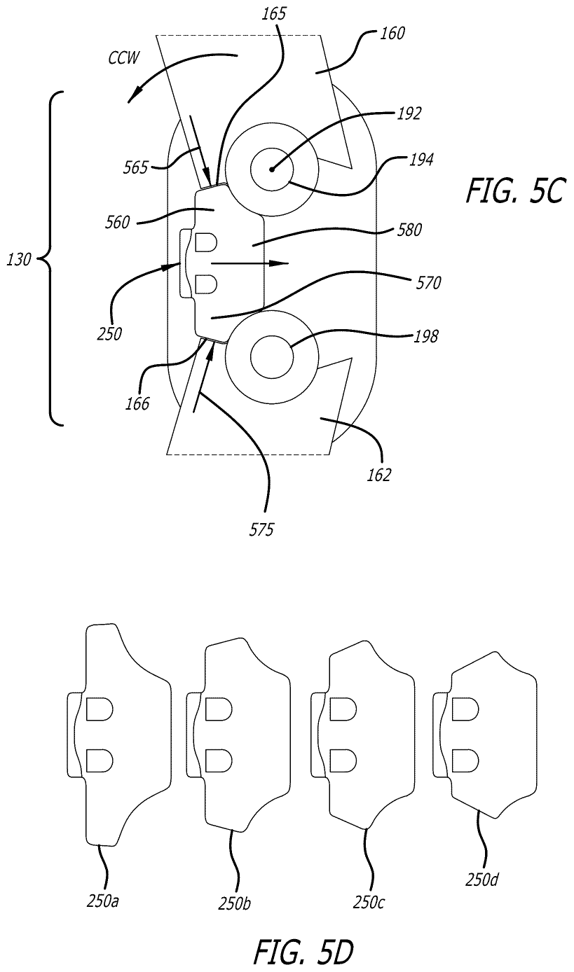

Referring now to FIG. 5A, a cross-sectional side view of the hinge 130 deploying a first type of extension stop component 240 to control the degree of extension allowed for the orthosis is shown. Herein, in response to extension of a body member within the orthosis 100, the first strut member 160 operates in manner consistent with movement of the first cuff of the orthosis 100 (movement in relation to the femur) to which the first strut member 160 is coupled. More specifically, the first strut member 160 pivots in a clockwise (CW) direction, causing a first edge 163 of the first strut member 160 to rotate in a CW direction around the first axis of rotation 192 (established by the first fastening element 194) until contacting a first (upper) limiter element 510 of the extension stop component 240 as shown by arrow 515.

Additionally, in the alternative, as shown in FIG. 5A, in response to extension of a body part within the orthosis 100, the second strut member 162 operates in manner consistent with movement of the second cuff 125 of the orthosis 100 of FIG. 1 (movement in relation to the tibia) to which the second strut member 162 is coupled. More specifically, the second strut member 162 pivots in a counterclockwise (CCW) direction, causing the first edge 164 of the second strut member 162 to rotate in a CCW direction around the second axis of rotation 196 (established by the second fastening element 198) until contacting a second (lower) extension limiter element 500 of the extension stop component 240 as shown by arrow 505.

Herein, the size (length) of the extension limiter elements 500 and 510 extending from a body 520 of the extension stop component 240 determines the prescribed degree of extension permitted by the central unit 150. As shown in FIG. 5B, different types of extension stop components 240a-240e that provide different degrees of controlled extension ranging from highest-to-lowest degrees of controlled extension is shown. For this embodiment, the illustrated extension stop components 240a-240e may be configured to provide different types of extension stop components with degrees of controlled extension such as extension stop components of sixty degrees (60°), forty-five degrees (45°), thirty degrees (30°), fifteen degrees (15°) and zero degrees (0°), respectively. The length of the extension limiter element(s) 500 and 510 is correlated to the degree of extension reduction and limitations on a range of motion.

Referring to FIG. 5C, a cross-sectional side view of the hinge 130 deploying a first type of flexion stop component 250 to control the degree of flexion allowed for the orthosis 100 of FIG. 1 is shown. Herein, in response to flexion of a body part within the orthosis 100, the first strut member 160 operates in manner consistent with movement of the first cuff of the orthosis 100 to which the first strut member 160 is coupled. More specifically, the first strut member 160 pivots in a CCW direction, causing the second edge 165 of the first strut member 160 to rotate in a CCW direction around the first fastening element 194 (and first axis of rotation 192) until contacting a first (upper) limiter element 560 of the flexion stop component 250 as shown by arrow 565.

Additionally, on in the alternative, as shown in FIG. 5C, the second strut member 162 operates in manner consistent with movement of the second cuff of the orthosis 100 to which the second strut member 162 is coupled. As a result, during a flexion activity, the second strut member 162 may pivot in a CW direction, causing the second edge 166 of the second strut member 162 to rotate in a CW direction around the second fastening element 198 until contacting a second (lower) extension limiter element 570 of the flexion stop component 250 as shown by arrow 575.

According to this embodiment, the size (length) of the extension limiter elements 560 and 570 extending from a body 580 of the flexion stop component 250 determines the prescribed degree of extension permitted by the central unit 150.

As shown in FIG. 5D, different types of flexion stop components 250a-250d are configured to provide different degrees of controlled flexion, ranging from lowest-to-highest degrees of controlled extension. For this illustration, the flexion stop components 250a-250d provide different degrees of controlled flexion such as fifteen degrees (15°), thirty degrees (30°), forty-five degrees (45°), and sixty degrees (60°), respectively. The length of the extension limiter element is negatively correlated to the degree of flexion. Also, it is contemplated that other flexion degree controls such as zero degrees (0°), seventy-five degrees (75°) and/or even ninety degrees (90°) may be utilized.

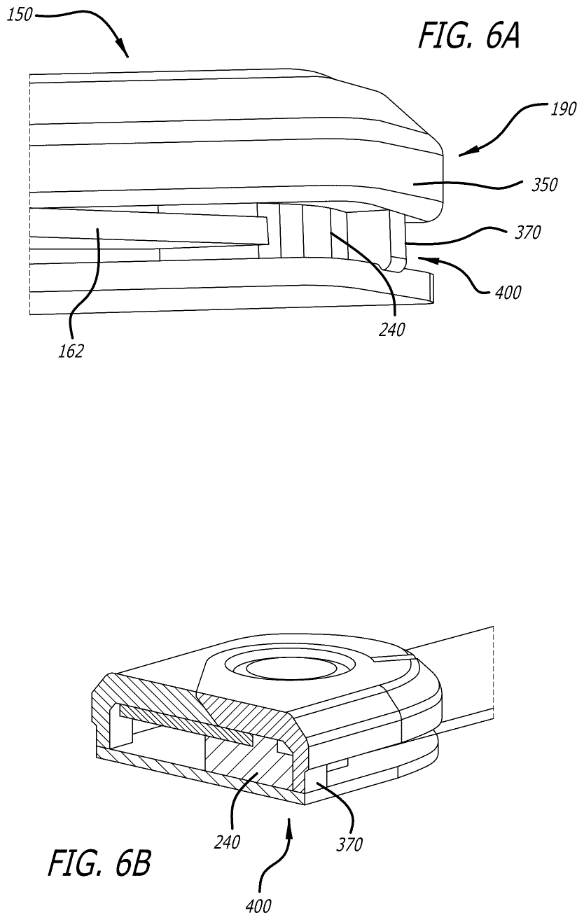

Referring to FIG. 6A, a perspective view of an exemplary embodiment of the first hinge cover 190 of the central unit 150, including the flange 370 extending from the outer edge 350 to assist in securing the extension stop component 240 within the central unit 150, is shown. In particular, when the first hinge cover 190 is rotated and placed into an open (or unlocked) state, the first set of stability notches formed along the first edge of the hinge front plate 200 are accessible to the extension stop component 240. Upon insertion of the extension stop component 240 into the entry area 400 between the first strut and the second strut member 162 and placement of the first hinge cover 190 into a closed or unlocked state (coupled to the second hinge cover 195), the flange 370 is positioned to overlap the entry area 400 to preclude the extension stop component 240 from being dislodged during use. A cross-sectional view of the first hinge cover 190 along with the flange 370 to represent the location of the flange in connection with the entry area 400 is shown in FIG. 6B.

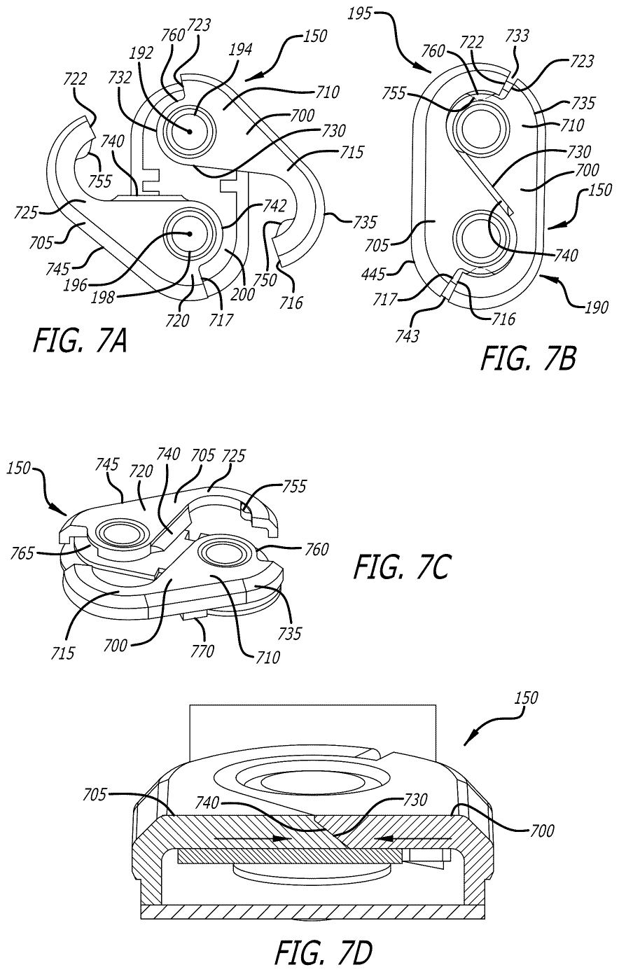

Referring now FIG. 7A, an overhead planar view of a second exemplary embodiment of the central unit 150 of FIGS. 1-2 placed into a partially open (or unlocked) state is shown. Similar to as described in FIGS. 3A-3C, the central unit 150 features a first hinge cover 700 and a second hinge cover 705. The first hinge cover 700 is mounted on the hinge front plate 200 and rotational about the first axis of rotation 192. The first axis of rotation 192 is colinear with the first fastening element 194. Similarly, the second hinge cover 705 is mounted on the hinge front plate 200 and rotational about the second axis of rotation 196. The second axis of rotation 196 is colinear with the second fastening element 198.

As shown, the first hinge cover 700 includes a first base component 710 and a first arm component 715 while the second hinge cover 705 includes a second base component 720 and a second arm component 725. Herein, the first base component 710 corresponds to a section of the first hinge cover 700 that surrounds an aperture adapted to receive the first fastening element 194. The first arm component 715, corresponding to a distal portion of the first hinge cover 700, is configured with a concave-shaped curvature to (i) partially align with an angular segment 742 of an inner edge 740 of the second hinge cover 705 and (ii) provide a gap 743 between a distal end 716 of the first arm component 715 for the first hinge cover 700 and a proximal end 717 of the second hinge cover 705 that connects the inner edge 740 of the second hinge cover 705 with its outer edge 745 as shown in FIG. 7B.

Similarly in construction, the second base component 720 corresponds to a section of the second hinge cover 705 that surrounds an aperture adapted to receive the second fastening element 198. The second arm component 725 corresponds to a section of the second hinge cover 705 having a concave-shaped curvature that (i) partially aligns with an angular segment 732 of an inner edge 730 of the first hinge cover 700 and (ii) provides a gap 733 between a distal end 722 for the second arm component 725 of the second hinge cover 705 and a proximal end 723 of the first hinge cover 700 that connects the inner edge 730 of the first hinge cover 700 with its outer edge 735 as shown in FIG. 7B. Herein, instead of both the inner edges 730 and 740 of the hinge covers 700 and 705 being set at a ninety degree pitch (e.g., flat from a side perspective), the inner edge 730 of the first hinge cover 700 is set to extend at a first prescribed angle (e.g., 30-60 degrees) while the inner edge 740 of the second hinge cover 705 is set to extend at a second prescribed angle that is supplementary to the first prescribed angle (e.g., 150-120 degrees). As a result, as shown in FIG. 7D, when the central unit 150 is placed into a closed (locked) state, the inner edge 730 of the first hinge cover 700 and the inner edge 740 of the second hinge cover 705 are placed into a layered relationship.

Referring back to FIGS. 7A-7B, the first gap 733 is formed between the proximal end 723 of the first hinge cover 700 and the distal end 722 of the second hinge cover 705 featuring indicia identifying a direction of movement needed from the first gap 733 to unlock the central unit 150. The second gap 743 is formed between the proximal end 717 of the second hinge cover 705 and the distal end 716 of the first hinge cover 700, which also features indicia identifying a direction of movement needed from the second gap 743 to unlock the central unit 150.

As further shown in FIGS. 7A-7B, the inner edge 730 of the first hinge cover 700 includes a detent (protrusion) 750 positioned toward at the distal end 716 of the first (concave) arm component 715 while the inner edge 740 of the second hinge cover 705 includes a detent (protrusion) 755 positioned toward at the distal end 722 of the second arm component 725. Given that the first hinge cover 700 and the second hinge cover 705 may feature identical constructions, the below discussion of the formulation of the detent 755 for the second hinge cover 705 is applicable to the formulation of the detent 750 for the first hinge cover 700.

More specifically, the detent 750 is configured to facilitate attachment of the first hinge cover 700 to the second hinge cover 705 while the detent 755 is configured to facilitate attachment of the second hinge cover 705 to the first hinge cover 700. More specifically, the detent 755 is sized to reside within a recessed area 760 formed with the first base component 710 of the first hinge cover 700 when the central unit 150 is placed in a closed (locked) state as shown in FIG. 7B. As the central unit 150 is placed into a closed (locked) state, prior to resting with the recessed area 760, the detent 755 comes into contact with an angular segment 732 of the inner edge 730 forming part of the first base component 710 and causes a slight increase in the curvature (e.g., flex or elongation of the second arm component 725) until the detent 755 clears an apex formed by the angular segment 732 and returns back to its “at rest” curvature (non-flex) to reside within the recessed area 760. The same operations occur for the detent 750 placed toward the distal end 716 of the first hinge cover 700. The same general architecture applies to detent 755 and recessed area 765.

Referring now to FIG. 7C, the outer edge 735 of the first hinge cover 700 includes a flange 770 positioned to overlap a portion of an entry area for the extension stop component 240 of FIG. 2 when the first hinge cover 700 is placed into a closed (locked) state. This overlapping flange 770 is adapted to preclude removal of the extension stop component 240, unless the central unit 150 is placed into an open (or unlocked) state in which both the first and second hinge covers 700 and 705 are rotated in a first direction (e.g., CCW) direction) concurrently. Similarly, the outer edge 745 of the second hinge cover 705 features a flange (not shown) positioned to overlap a portion of an entry area for the flexion stop component 250 of FIG. 2. This overlapping flange is adapted to preclude removal of the flexion stop component 250 unless the central unit 150 is placed into an open (or unlocked) state in which both the first and second hinge covers 700 and 705 are rotated in the first direction concurrently.

FIG. 8A-8E are translucent views of the hinge 130 with the central unit 150 deploying extension stop components with different extension ranges. Herein, for illustrative sake, the extension stop components 240 deployed within the central unit 150 may include different types of extension stop components that provide different degrees of controlled extension such as a first (0°) extension stop component 240a, a second (15°) extension stop component 240b, a third (30°) extension stop component 240c, a fourth (45°) extension stop component 240d, and/or a fifth (60°) extension stop component 240e. The extension stop component 240a . . . or 240e is placed within the central unit 150 residing in an open (unlocked) state and securely retained within the central unit 150 when the central unit 150 is placed into a closed (locked) state.

FIGS. 9A-9B are translucent views of the hinge 130 with the central unit 150 deploying flexion stop-components with different flexion ranges. Herein, for illustration, the flexion stop components may include different types of flexion stop components that provide different degrees of controlled flexion such as a first (15°) flexion stop component 250a, a second (30°) flexion stop component 250b, a third (45°) flexion stop component 250c, and a fourth (60°) flexion stop component 250d. The flexion stop component 250a . . . or 250d is placed within the central unit 150 residing in an open (unlocked) state and securely retained within the central unit 150 when the central unit 150 is placed into a closed (locked) state.

### V. Hinge Operability

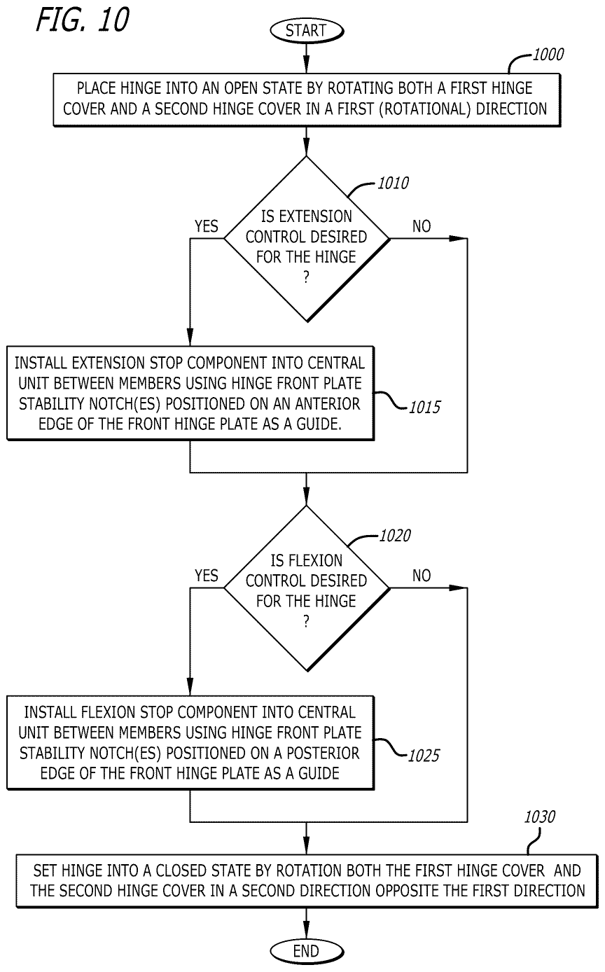

Referring now to FIG. 10, an illustrative method of operation for setting the hinge into an operational state with a prescribed degree of extension and/or flexion is shown. First, the hinge is placed into an open (or unlocked) state by rotating both a first hinge cover and the second hinge cover of the hinge in a first rotational direction (operation 1000). The rotation of the hinge covers places the central unit (and hinge) into an open (or unlocked) state and laterally exposes a middle area (entry areas) of the hinge between the hinge front plate and the hinge back plate. Thereafter, if extension control is desired, an extension stop component is installed with a first entry area between ends of the members (operations 1010-1015). An anterior edge of the hinge front plate features a stability notch that operates as a guide for placement of the extension stop component. If flexion control is desired, a flexion stop component is installed with a second entry area between other ends of the struts (operations 1020-1025). A posterior edge of the hinge front plate features one or more stability notches that operates as a guide for placement of the flexion stop component.

After installation of the extension stop component and/or the flexion stop component, the central unit (and hinge) is placed into a closed (locked) state by rotating both the first hinge cover and the second hinge cover of the hinge in a second rotational direction that is angularly opposite the first rotational direction (operation 1030). Thereafter, the hinge is set to operate and control extension and/or flexion movement by the patient wearing the orthosis.

### VI. Second Hinge—General Architecture

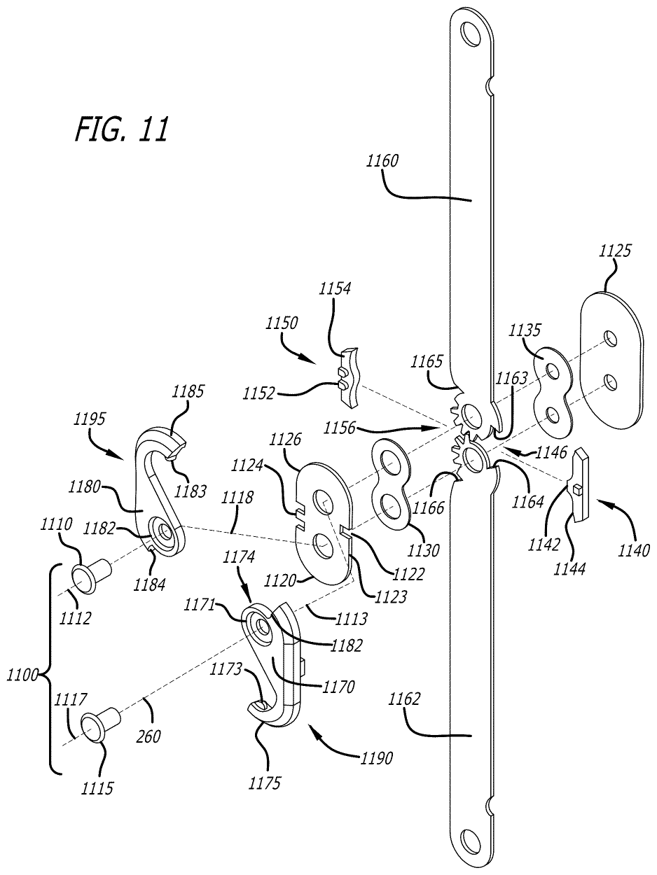

Referring now to FIG. 11, an illustrative embodiment of mechanical components of the hinge 130 of FIG. 1 is shown, which include a central unit 1100 and a pair of geared strut members 1160 and 1162. For this embodiment, the central unit 1100 includes a first and second fastening elements 1110 and 1115; the first and second hinge covers 1190 and 1195; a hinge front plate 1120; a hinge back plate 1125; a first washer 1130 positioned between the hinge front plate 1120 and geared strut members 1160 and 1162; a second washer 235 positioned between the hinge back plate 1125 and the geared strut members 1160 and 1162; an optional, removable extension stop component 1140; and/or an optional, removable flexion stop component 1150. A coupling path 1113 illustrates an attachment sequence for the first fastening element 1110 to form the first axis of rotation 1112 while a second coupling path 1118 illustrates an attachment sequence for the second fastening element 1115 to form the second axis of rotation 1117.

More specifically, the first hinge cover 1190 includes a first base component 1170 and a first arm component 1175 while the second hinge cover 1195 includes a second base component 1180 and a second arm component 1185. Herein, according to this embodiment of the disclosure. the first hinge cover 1190 is rotationally coupled to the hinge front plate 1120 by insertion of the first fastening element 1110 into a recessed aperture 1171 formed within the first base component 1170. An end section of the first base component 1170 features a recessed area 1174 sized to receive a detent (protrusion) 1183 situated within the second arm component 1185 of the second hinge cover 1195. The first arm component 1175 of the first hinge cover 1190 includes a first detent (protrusion) 1173, which is sized for retention within a recessed area 1184 of the second hinge cover 1195.

Similarly, the second hinge cover 1195 is rotationally coupled to the hinge front plate 200 by insertion of the second fastening element 1115 into a recessed aperture 1182 formed within the second base component 1180. An end section of the second base component 1180 features the recessed area 1184 sized to receive the first detent 1173 situated within the first arm component 1175 of the first hinge cover 1190. The second arm component 1185 of the second hinge cover 1195 includes the detent (protrusion) 1183, which is sized for retention within the recessed area 1174 of the first hinge cover 1190.

Referring still to FIG. 11, the hinge front plate 1120 is configured with a first set of stability notches (e.g., first stability notch 1122) may be formed along a first edge 1123 of the hinge front plate 1120. The first stability notch 1122 is formed to engage with a first stability element 1142 positioned on a side surface 1144 of the extension stop component 1140. This engagement is designed to maintain horizontal and vertical alignment of the extension stop component 1140 during usage of the orthosis 100. Additionally, a second set of stability notches (e.g., second and third stability notches 1124) may be formed along a second edge 1126 of the hinge front plate 1120. These stability notches 1124 are formed to engage with a corresponding set of stability elements 1152 positioned on a side surface 1154 of the flexion stop component 1150. As shown, the number of stability elements 1142 positioned on the extension stop component 1140 may differ from the number of stability elements 1152 positioned on the flexion stop component 1150 for easy identification as to whether the stop component is designed to control extension or flexion.

The extension stop component 1140 is positioned to reside within a first (anterior) spacing 1146 formed between first edges 1163 and 1164 of the geared strut members 1160 and 1162, with the first stability element 1142 being positioned to engage with the first stability notch 1122 when the extension stop component 1140 is placed within the first spacing 1146. Similar in construction, the flexion stop component 1150 is formed to reside within a second (posterior) spacing 1156 formed between second edges 1165 and 1166 of the geared strut members 1160 and 1162, with the set of stability elements 1152 being positioned to engage with the stability notches 1124 when the flexion stop component 1150 is placed within the second spacing 1156.

During extension, when viewed from a lateral-to-medial perspective for a right leg, the central unit 1100 remains fixed while both the first (geared) strut member 1160 and the second (geared) strut member 1160 to move concurrently at the same rate. Hence, forward (CCW) movement of the second (geared) strut member 1162 occurs until the first edge 1164 of the second strut member 1162 engages the extension stop component 1140, which is sized to allow a prescribed degree of extension. Also, forward (CW) movement of the first (geared) strut member 1160 occurs until the first edge 1163 engages with the extension stop component 1140. Similarly, during flexion, rearward (CW) movement of the second strut member 1162 occurs until the second edge 1166 of the second strut member 1162 engages with the flexion stop component 1150, which is sized to allow a prescribed degree of flexion. Also, rearward (CCW) movement of the first (geared) strut member 1160 occurs until the second edge 1165 engages with the flexion stop component 1150.

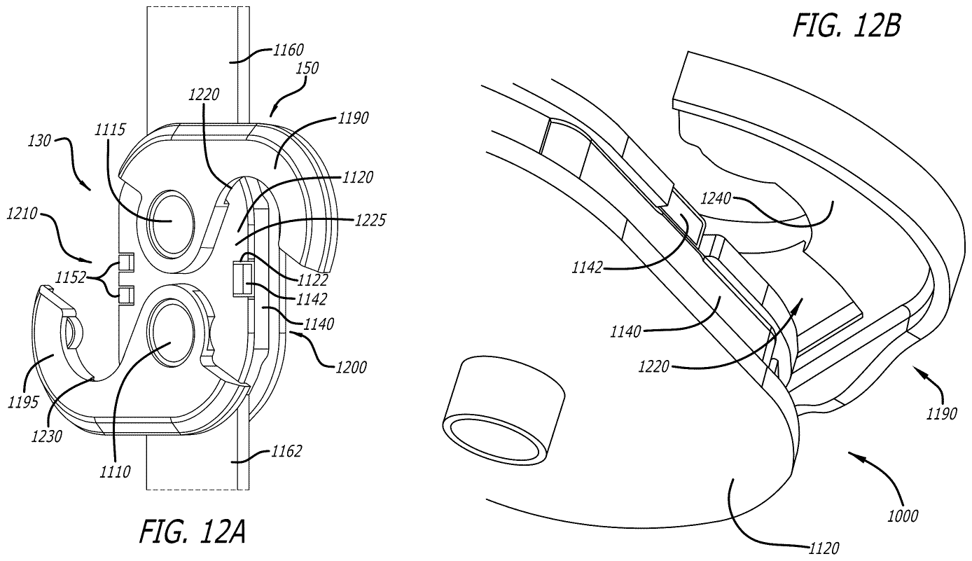

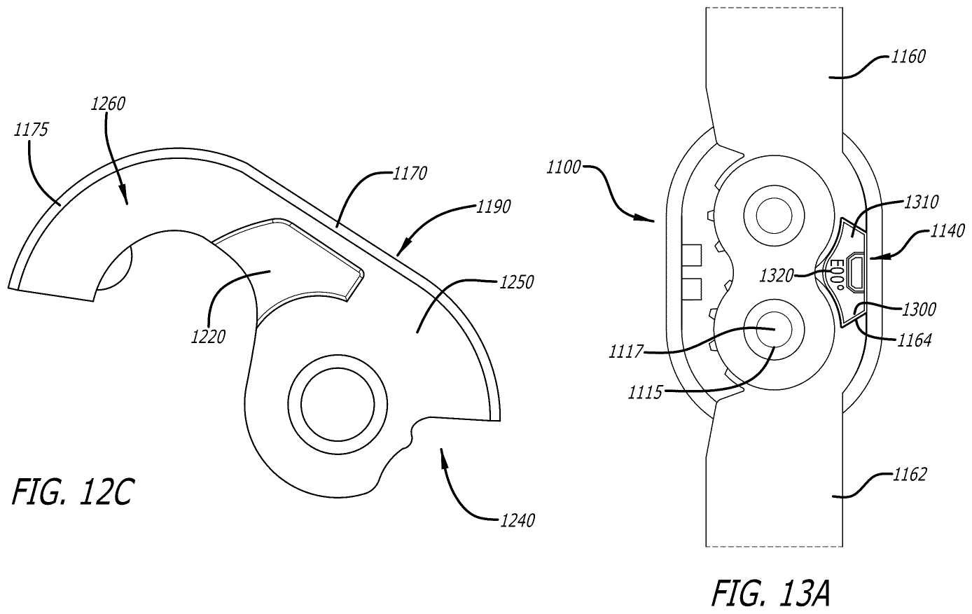

Referring now to FIG. 12A, a perspective view of an illustrative embodiment of the hinge 130 of FIG. 11 oriented in a resting (non-flexed) state and the central unit 1100 placed in an open (or unlocked) state. To retain the central unit 1100 to the geared strut members 1160 and 1162, the fastening elements 1110 and 1115 are placed through apertures formed in the hinge covers 1190 and 1195, the hinge front plate 1120, geared strut members 1160 and 1162, and the hinge back plate 1125. Entry areas 1200 and 1210 are formed between geared ends of the geared strut members 1160 and 1162 to receive the stop component 1140 and/or the stop component 1150. The hinge covers 1190 and 1195 are rotated to expose the entry areas 1200 and 1210 for insertion of the stop components (e.g., extension stop component 1140) when desired.

As shown, the first stability element 1142 of the extension stop component 1140 engages with the first stability notch 1122 of the hinge front plate 1120. A backside of the first hinge cover 1190 features a recessed area 1220 that is positioned to reside over the first stability element 1142 extending from a top surface 1225 of the hinge front plate 1120. This recessed area 1220 is designed to restrain the first stability element 1142 when the first hinge cover 1190 is placed into a closed (locked) state as shown by the hinge cover positioning in FIGS. 4B-4C, thereby preventing or substantially mitigating anterior movement of the extension stop component 1140. Additionally, a backside of the second hinge cover 1195 features a recessed area 1230, which is positioned to reside over the set of stability elements 1152 positioned on the flexion stop component (not shown). This prevents posterior movement of the flexion stop component when the second hinge cover 1195 is placed into a closed (locked) state similar to FIGS. 4B-4C.

As described above and similarly illustrated in FIGS. 3A-3C, the detent 1173 is configured to facilitate attachment of the first hinge cover 1190 to the second hinge cover 1195 while the other detent 1183 is configured to facilitate attachment of the second hinge cover 1195 to the first hinge cover 1190. In particular, as the central unit 1100 is placed into a closed (locked) state, prior to resting with a recessed area, the detent 1173 comes into contact with an angular segment of an inner edge forming part of the second base component 1170, which causes a slight increase in the curvature of the first arm component 1175 (e.g., flex or elongation) until the detent 1173 clears an apex formed by the angular segment and returns back to its “at rest” curvature (non-flex state) to reside within the recessed area to “snap” into a closed (locked) state. The same operations occur for the detent 1183 placed toward a distal end of the second hinge cover 1195.

Referring to FIG. 12B, an upward perspective view of an illustrative embodiment of a backside 1240 of the first hinge cover 1190 of the central unit 1100 of FIG. 12A is shown, where the first hinge cover 1190 is transitioning from an unlocked state to a locked state. As a result, the recessed area 1220 is arranged to cover the first stability element 1142 of the extension stop component 1140 and prevent movement of the extension stop component 1140. A similar architecture is configured for the second hinge cover 1195 of FIG. 12A, where the recessed area 1230 is configured to restrain the set of stability elements 1152.

An illustrative planar view of the backside 1240 of the first hinge cover 1190 is shown in FIG. 12C. The recessed area 1220 is positioned on a backside segment 1250 of the base component 1170 while a backside segment 1260 of the first arm component 1175 features a greater height to account for the spacing between the backside 1240 of the first hinge cover 1190 and the top surface 1225 of the hinge front plate 1120.

Referring now to FIG. 13A, a cross-sectional side view of the hinge 130 with the central unit 1100 is shown, where the central unit 1100 is configured to deploy a zero degree (0°) extension stop component 1140e (see FIG. 13C) to control the degree of extension allowed for the orthosis. Herein, in response to extension of a body member within the orthosis 100, the first strut member 1160 operates in manner consistent with movement of the first cuff of the orthosis 100 (movement in relation to the femur) to which the first strut member 1160 is coupled. More specifically, if the first strut member 1160 pivots in a clockwise (CW) direction around the first axis of rotation 1112, it would cause a first edge 1163 of the first strut member 1160 to rotate until contacting a first (upper) limiter element 1310 of the extension stop component 1140.

Additionally, in the alternative, as shown in FIG. 13A, in response to extension of a body part within the orthosis 100, the second strut member 1162 operates in manner consistent with movement of the second cuff 125 of the orthosis 100 of FIG. 1 (movement in relation to the tibia) to which the second strut member 1162 is coupled. More specifically, the second strut member 1162 pivots in a counterclockwise (CCW) direction, causing the first edge 1164 of the second strut member 1162 to rotate in a CCW direction around the second axis of rotation 1117 (established by the second fastening element 1115) until contacting a second (lower) extension limiter element 1300 of the extension stop component 1140

Herein, the size (length) of the extension limiter elements 1300 and 1310 extending from a body 1320 of the extension stop component 1140 and angular orientations of their lateral surfaces 1345 and 1355 determine the prescribed degree of extension permitted by the central unit 1100. As shown in FIG. 13B, the geared strut members 1160 and 1162 are illustrated with gear segments 1330 and 1340 positioned at the ends of the geared strut members 1160 and 1162, respectively. The teeth 1335/1345 of the gear segments 1330/1340 are configured to interlock with each other to retain alignment of the central unit 1100 with the geared strut members 1160 and 1162.

As shown in FIG. 13C, different types of extension stop components 1140a -1140e that provide different degrees of controlled extension ranging from highest-to-lowest degrees of controlled extension is shown. For this embodiment, the illustrated extension stop components 1140a -1140e provide extension degree controls of sixty degrees (60°), forty-five degrees (45°), thirty degrees (30°), fifteen degrees, (15 °) and zero degrees (0°), respectively. The length and sidewall angular orientation of the extension limiter element(s) 1300 and 1310 is correlated to the degree of extension reduction and limitations on a range of motion.

Referring to FIG. 13D, a cross-sectional side view of the hinge 130 with the central unit 1100 is shown, where the central unit 1100 is configured to deploy a first type of flexion stop component 250 (e.g., 45° flexion stop component) to control the degree of flexion allowed for the orthosis 100 of FIG. 1 is shown. Herein, in response to flexion movement of a body part within the orthosis 100, the first strut member 1160 operates in manner consistent with movement of the first cuff of the orthosis 100 to which the first strut member 1160 is coupled. More specifically, if the first strut member 1160 pivots in a counter-clockwise (CCW) direction around the first axis of rotation 1112, it would cause a first edge 1165 of the first strut member 1160 to rotate until contacting a surface 1355 of the first (upper) limiter element 1350 of the flexion stop component 1150.

For flexion movement, however, the second strut member 1162 operates in manner consistent with movement of the second cuff of the orthosis 100 to which the second strut member 1162 is coupled. As a result, during a flexion activity, the second strut member 1162 may pivot in a CW direction, causing the second edge 1166 of the second strut member 1162 to rotate in a CW direction around the second fastening element 1115 until contacting a surface 1365 of the second (lower) extension limiter element 1360 of the flexion stop component 250.

According to this embodiment, the size (length) of the extension limiter elements 1350 and 1360 extending from a body 1370 of the flexion stop component 1150 and angular orientations of their lateral surfaces 1355 and 1365 determines the prescribed degree of flexion permitted by the central unit 1100.

Herein, the size (length) and angular orientation of the extension limiter elements 1300 and 1310 extending from a body 1320 of the extension stop component 1140 determine the prescribed degree of extension permitted by the central unit 1100.

As shown in FIG. 13E, different types of flexion stop components 1150a -1150f are configured to provide different degrees of controlled flexion, ranging from lowest-to-highest degrees of controlled extension. For this illustration, the flexion stop components 1150a -1150f provide flexion degree controls of zero degrees (0°), thirty degrees (30°), forty-five degrees (45°), and sixty degrees (60°), seventy-five degrees (75°), and ninety degrees (90°), respectively. The length of the extension limiter element is negatively correlated to the degree of flexion.

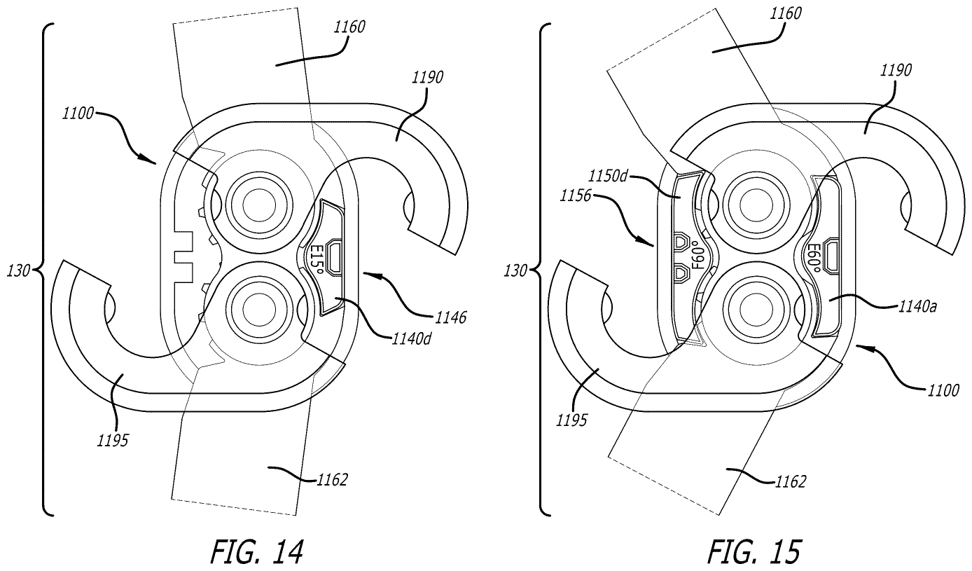

Referring now to FIG. 14, a translucent view of the hinge 130 with the central unit 1100 deploying the extension stop component 1140 within the first spacing 1146. As shown, the extension stop component 1140 may include a fifteen degrees (15°) extension stop component 1140b. The extension stop component 1140b is placed within the central unit 1100 residing in an open (unlocked) state as shown, and securely retained within the central unit 1100 when the central unit 1100 is placed into a closed (locked) state upon CW rotation of the first and second hinge covers 1190 and 1195.

Referring to FIG. 15, a translucent view of the hinge 130 with the central unit 1100 deploying the flexion stop component 1150 within the second (posterior) spacing 1156. As illustrated, the flexion stop component 1150 may include a sixty degrees (60°) flexion stop component 1150d. The flexion stop component 1150d is placed within the central unit 1100 residing in an open (unlocked) state as shown, and securely retained within the central unit 1100 when the central unit 1100 is placed into a closed (locked) state upon CW rotation of the first and second hinge covers 1190 and 1195. An extension stop component may be installed separate from or in combination with a flexion stop component, as the extension stop component 1140 with sixty degrees (60°) extension stop component is installed herewith.

In the foregoing description, the invention is described with reference to specific exemplary embodiments thereof. However, it will be evident that various modifications and changes may be made thereto without departing from the broader spirit and scope of the invention as set forth in the appended claims.

What is claimed is:

1. A hinge comprising:
a hinge front plate;
a first hinge cover rotationally coupled to the hinge front plate, the first hinge cover includes a first base component and a first arm component extending from the first base component; and
a second hinge cover rotationally coupled to the hinge front plate, the second hinge cover includes a second base component and a second arm component extending from the second base component,
wherein concurrent rotational movement of the first hinge cover and the second hinge cover in a first rotational direction places a central unit, including the first hinge cover and the second hinge cover, into a locked state and concurrent rotational movement of the first hinge cover and the second hinge cover in a second rotational direction places the central unit into an unlocked state.

2. The hinge of claim 1 further comprising:
a removable extension stop component positioned within a first spacing between the hinge front plate and a rotational strut to restrict movement of the rotational strut, the removable extension stop component includes a first stability element being positioned to engage with a first stability notch formed in the hinge front plate when the removable extension stop component is placed within the first spacing.

3. The hinge of claim 2 further comprising:
a removable flexion stop component positioned within a second spacing between the hinge front plate and the rotational strut to restrict flexion movement of the rotational strut, the removable flexion stop component includes one or more stability elements being positioned to engage with at least a stability notch formed in the hinge front plate when the removable flexion stop component is placed within the second spacing.

4. The hinge of claim 1, wherein a first region of the first base component of the first hinge cover includes a first recess, a first region of the first arm component of the first hinge cover includes a first detent, a first region of the second base component of the second hinge cover includes a second recess, a first region of the second arm component of the second hinge cover includes a second detent.

5. The hinge of claim 4 being set into the locked state when the first hinge cover and the second hinge cover are rotated so that the first detent of the first arm component is positioned with the second recess of the second base component and the second detent of the second arm component is positioned with the first recess of the first base component.

6. The hinge of claim 2, wherein a first region of the first base component of the first hinge cover comprises a recessed area positioned on a backside of the first hinge cover, the recessed area is sized with a height to restrain the first stability element and prevent movement of the first stability element when the first hinge cover is placed into the locked state.

7. The hinge of claim 1, wherein the second arm component corresponds to a section of the second hinge cover having a concave-shaped curvature that (i) partially aligns with a convex-shaped segment of an inner edge of the first base component of the first hinge cover and (ii) provides a gap between a distal end of the second hinge cover at the second arm component and a proximal end of the first hinge cover that connects the inner edge with an outer edge of the first base component.

8. The hinge of claim 7, wherein the first arm component corresponds to a section of the first hinge cover having a concave-shaped curvature that (i) partially aligns with a convex-shaped segment of an inner edge of the second base component of the second hinge cover and (ii) provides a gap between a distal end of the first hinge cover at the first arm component and a proximal end of the second hinge cover that connects the inner edge with an outer edge of the second base component.

9. The hinge of claim 4, wherein the first detent of the first arm component of the first hinge cover is positioned to contact an angular segment of an inner edge forming part of the second base component of the second hinge cover to cause a flex of the first arm component until the first detent clears the angular segment and returns back to a non-flexed length to reside within the second recess of the second base component when the first hinge cover and the second hinge cover are placed into the locked state.

10. The hinge of claim 1 further comprising:
a hinge back plate;
a first strut member rotationally coupled to the first hinge cover and interposed between the hinge front plate and the hinge back plate; and
a second strut member rotationally coupled to the second hinge cover and interposed between the hinge front plate and the hinge back plate,
wherein the first strut member and the second strut member operate in concert as geared struts.

11. The hinge of claim 10 further comprising:
an extension stop component positioned to reside within a first spacing formed between a first edge of the first strut member and a first edge of the second strut member, the extension stop component including a first stability element being positioned to engage with a first stability notch formed in the hinge front plate when the extension stop component is placed within the first spacing.

12. The hinge of claim 11, wherein the first hinge cover includes a flange extending from an outer edge of the first base component to assist in securing the extension stop component.

13. The hinge of claim 10 further comprising:
a flexion stop component positioned to reside within a spacing formed between an edge of the first strut member and an edge of the second strut member, the flexion stop component including one or more stability elements being positioned to engage with one or more stability notches formed in the hinge front plate when the flexion stop component is placed within the spacing.

14. An orthosis, comprising:
a sleeve;
a first strut member integrated at least partially within the sleeve;
a second strut member integrated at least partially within the sleeve; and
a central unit pivotally coupled to the first strut member and the second strut member, the central unit including
a hinge front plate,
a first hinge cover rotationally coupled to the hinge front plate, the first hinge cover includes a first base component and a first arm component extending from the first base component, and
a second hinge cover rotationally coupled to the hinge front plate, the second hinge cover includes a second base component and a second arm component extending from the second base component,
wherein concurrent rotational movement of the first hinge cover and the second hinge cover in a first rotational direction places the central unit into a locked state and concurrent rotational movement of the first hinge cover and the second hinge cover in a second rotational direction places the central unit into an unlocked state.

15. The orthosis of claim 14, wherein a first region of the first base component of the first hinge cover includes a first recess, a first region of the first arm component of the first hinge cover includes a first detent, a first region of the second base component of the second hinge cover includes a second recess, a first region of the second arm component of the second hinge cover includes a second detent.

16. The orthosis of claim 15, wherein the central unit is set into the locked state when the first hinge cover and the second hinge cover are rotated so that the first detent of the first arm component is positioned with the second recess of the second base component and the second detent of the second arm component is positioned with the first recess of the first base component.

17. The orthosis of claim 14, wherein the second arm component corresponds to a section of the second hinge cover having a concave-shaped curvature that (i) partially aligns with a convex-shaped segment of an inner edge of the first base component of the first hinge cover and (ii) provides a gap between a distal end of the second hinge cover at the second arm component and a proximal end of the first hinge cover that connects the inner edge with an outer edge of the first base component.

18. The orthosis of claim 17, wherein the first arm component corresponds to a section of the first hinge cover having a concave-shaped curvature that (i) partially aligns with a convex-shaped segment of an inner edge of the second base component of the second hinge cover and (ii) provides a gap between a distal end of the first hinge cover at the first arm component and a proximal end of the second hinge cover that connects the inner edge with an outer edge of the second base component.

19. The orthosis of claim 15, wherein the first detent of the first arm component of the first hinge cover is positioned to contact a raised segment formed on an inner edge forming part of the second base component of the second hinge cover to cause a flex of the first arm component until the first detent clears the raised segment and returns back to a non-flexed length to reside within the second recess of the second base component when the first hinge cover and the second hinge cover are placed into the locked state.

20. The orthosis of claim 14 further comprising a hinge back plate, wherein
the first strut member is rotationally coupled to the first hinge cover and interposed between the hinge front plate and the hinge back plate, and
the second strut member is rotationally coupled to the second hinge cover and interposed between the hinge front plate and the hinge back plate.

21. The orthosis of claim 20 further comprising:
an extension stop component positioned to reside within a first spacing formed between a first edge of the first strut member and a first edge of the second strut member, the extension stop component including a first stability element being positioned to engage with a first stability notch formed in the hinge front plate when the extension stop component is placed within the first spacing.

22. The orthosis of claim 21, wherein the first hinge cover includes a flange extending from an outer edge of the first base component of the first hinge cover to assist in securing the extension stop component.

23. The orthosis of claim 21 further comprising:
a flexion stop component positioned to reside within a spacing formed between an edge of the first strut member and an edge of the second strut member, the flexion stop component including one or more stability elements being positioned to engage with one or more stability notches formed in the hinge front plate when the flexion stop component is placed within the spacing.

24. The orthosis of claim 21, wherein the extension stop component is prevented from being removed when the central unit is placed into the locked state and the extension stop component is able to be removed from the central unit when the central unit is placed into the unlocked state.

25. The orthosis of claim 14, wherein the sleeve comprises (1) a plurality of pockets sized to receive a first cuff corresponding to a sheet of hardened material positioned above a knee area and a second cuff including a sheet of hardened material positioned below the knee area, and (2) a plurality of channels to substantially integrate interconnects coupled between the first cuff and the second cuff, where the interconnects can be tightened or loosened by a rotating cable tensioning mechanism accessible through the sleeve.

26. A hinge comprising:
a plurality of geared struts including a first geared strut and a second geared strut; and
a central unit including
first hinge cover including a first base component and a first arm component extending from the first base component, wherein the first hinge cover is configured to rotate in a first rotational direction and a second rotational direction opposite to the first rotational direction;
a second hinge cover including a second base component and a second arm component extending from the second base component, wherein the second hinge cover is configured to rotate, independent of the first hinge cover, in the first rotational direction and the second rotational direction, and
one or more stop components removably positioned within the central unit to restrict movement of the second geared strut,
wherein concurrent rotational movement of the first hinge cover and the second hinge cover in the first rotational direction places the central unit into a locked state to secure positioning of the one or more stop components and concurrent rotational movement of the first hinge cover and the second hinge cover in the second rotational direction places the central unit into an unlocked state to allow for removal of the one or more stop components from the central unit.
